(19) United States
(12) Patent Application Publication
(10) Pub. No.: US 2026/0182860 A1
(43) Pub. Date: Jul. 2, 2026

(54) BREATH DETECTION METHOD, PROGRAM, AND SENSOR MODULE

(71) Applicant: PANASONIC HOUSING SOLUTIONS CO., LTD., Osaka (JP)

(72) Inventors: Takuya HAYASHI, Osaka (JP); Masaya NAKATANI, Hyogo (JP); Tsuguhide IOKI, Hyogo (JP)

(21) Appl. No.: 19/127,335

(22) PCT Filed: Nov. 9, 2023
(86) PCT No.: PCT/JP2023/040381

§ 371 Date: May 5, 2025

(30) Foreign Application Priority Data
Nov. 29, 2022 (JP) 2022-190474

(51) Int. Cl. A61B 5/087; A61B 5/08; G16H 40/60

(52) U.S. Cl. CPC A61B 5/0873; A61B 5/0803; G16H 40/60

(57) ABSTRACT

A breath detection method includes a temperature control step and an instruction information output step. The temperature control step includes controlling a temperature of a sensitive unit to cause the temperature of the sensitive unit to change in a predetermined temperature change pattern with time during a measuring period. The instruction information output step includes outputting instruction information to instruct the subject how to blow his or her breath toward the sensitive unit during an exhalation period which is set in accordance

### TECHNICAL FIELD

The present disclosure generally relates to a breath detection method, a program, and a sensor module. More particularly, the present disclosure relates to a breath detection method, a program, and a sensor module, all of which are designed to detect, by measuring, the properties of breath or the state of a target gas included in the breath.

### BACKGROUND ART

Patent Literature 1 discloses a breath detection device including a pressure detector, a temperature sensor, and a determiner. The pressure detector detects the pressure received at a breath blower to output a signal. The temperature sensor detects the temperature at the breath blower. The determiner compares signals supplied from the pressure detector and the temperature sensor with their respective criteria, thereby determining, based on the results of the comparisons, whether a person has blown his or her breath toward the breath blower.

When either the properties of breath or the state of a target gas included in the breath is going to be detected, the subject should blow his or her breath toward a sensitive unit appropriately. Otherwise, his or her breath would not be measured properly.

### CITATION LIST

### Patent Literature

Patent Literature 1: JP 2015-232654 A

### SUMMARY OF INVENTION

An object of the present disclosure is to provide a breath detection method, a program, and a sensor module, all of which are designed to reduce the chances of measuring a subject's breath while allowing the subject to blow his or her breath inappropriately toward a sensitive unit.

A breath detection method according to an aspect of the present disclosure includes a temperature control step and an instruction information output step. The temperature control step includes controlling a temperature of a sensitive unit to cause the temperature of the sensitive unit to change in a predetermined temperature change pattern with time during a measuring period. The instruction information output step includes outputting instruction information to instruct a subject how to blow a breath toward the sensitive unit during an exhalation period. The exhalation period is set in accordance with the temperature change pattern.

A program according to another aspect of the present disclosure is designed to cause a computer system to perform the breath detection method described above.

A sensor module according to still another aspect of the present disclosure is connectible to an electronic device. The electronic device includes a user interface and instructs a subject how to blow a breath toward a sensitive unit. The sensor module includes: the sensitive unit; a temperature adjustment unit to heat and/or cool the sensitive unit; and a housing which houses the sensitive unit and the temperature adjustment unit and is designed to be removably attached to the electronic device.

### BRIEF DESCRIPTION OF DRAWINGS

FIG. 1 is a schematic block diagram of a breath detection system including a sensor module according to an exemplary embodiment of the present disclosure and an electronic device to which the sensor module is connected;

FIG. 2 is a front view illustrating a state where the sensor module is attached to an electronic device;

FIG. 3 schematically illustrates a sensitive unit included in the sensor module;

FIG. 4 schematically illustrates a state of the sensitive unit before the sensitive unit absorbs molecules to detect and a state of the sensitive unit after the sensitive unit has absorbed the molecules to detect;

FIG. 5 is a graph showing how the resistance value of a positive characteristic sensitive element included in the sensitive unit changes with time in a situation where the temperature of the sensitive unit has been changed;

FIG. 6 is a flowchart showing the procedure in which the breath detection system performs a measuring operation; and

FIG. 7 is a graph showing how the temperature and resistance value change with time during a measuring period of the sensitive unit.

### DESCRIPTION OF EMBODIMENTS

An embodiment of the present disclosure will now be described with reference to the accompanying drawings as needed. Note that the embodiment to be described below is only an exemplary one of various embodiments of the present disclosure and should not be construed as limiting. The relative positions of respective constituent elements in upward, downward, rightward, and leftward directions are supposed to be defined as shown on the drawings unless otherwise stated. The drawings to be referred to in the following description of embodiments are all schematic representations. Thus, the ratio of the dimensions (including thicknesses) of respective constituent elements illustrated on the drawings does not always reflect their actual dimensional ratio. Also, the dimensional ratio of the respective elements is not limited to the ratio shown on the drawings.

As for each of the materials exemplified in the following description, the material may be used either by itself or in combination with at least one more of the materials, whichever is appropriate, unless otherwise stated. As for the content of each component included in a composition, if there are multiple substances corresponding to that component in the composition, the content of the component means the total content of those substances included in the composition, unless otherwise stated.

### Embodiment

### (1) Overview

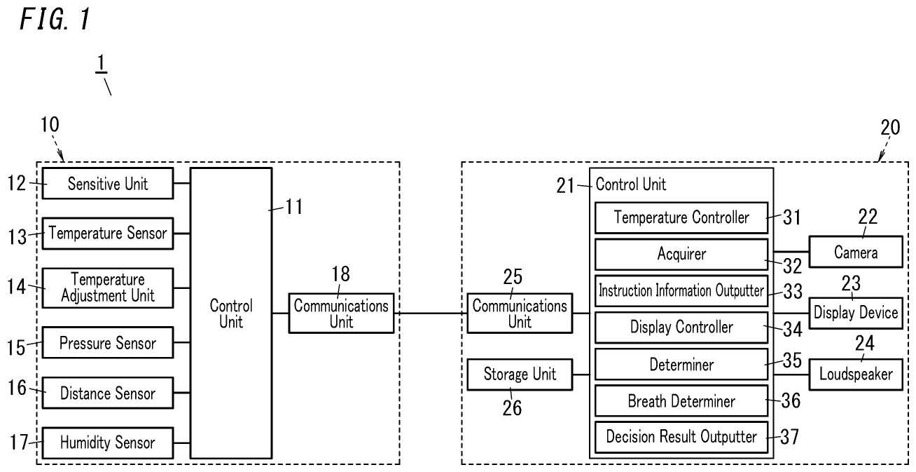

FIG. 1 illustrates a schematic system configuration for a breath detection system 1 according to an exemplary embodiment.

The breath detection system 1 includes a sensor module 10 and an electronic device 20 to which the sensor module 10 is attached. The breath detection system 1 detects either the properties of the breath that a subject has blown toward a sensitive unit 12 included in the sensor module 10 or the state of a target gas included in the breath. As used herein, the “properties of the breath or the state of a target gas included in the breath” to be detected by the breath detection system 1 may refer to, for example, whether or not the breath has any odor (such as bad breath) and a state of the target gas as the target of measurement, more specifically, the concentration of alcohol in the breath, the type and composition of molecules included in the breath which may be used to identify a particular person, and the type and composition of molecules included in the breath which have been produced due to the onset of a particular disease. If the breath detection system 1 is designed to detect the odor of the breath as a property of the breath, then the odor of the target to detect may include volatile organic compounds (VOCs) such as such as methane, acetone, ethanol, isoprene, ethane, pentane, benzaldehyde, nonanal, and pyrrole and odor molecules such as ammonia.

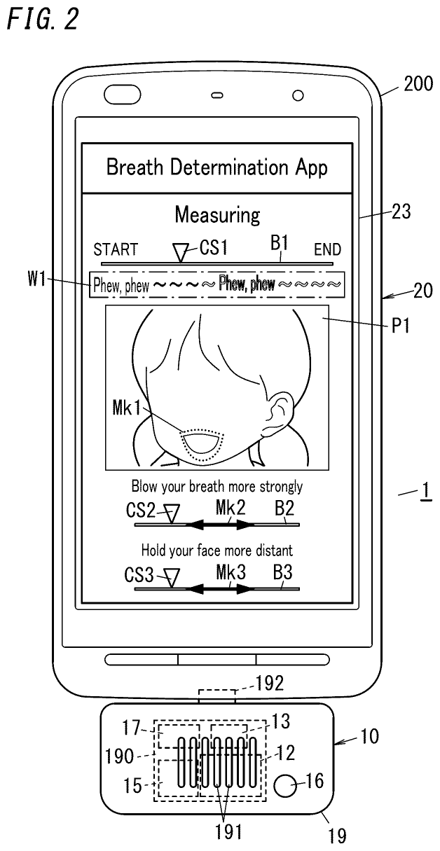

The sensor module 10 is connectible to the electronic device 20 including a user interface. The sensor module 10 includes a sensitive unit 12, a temperature adjustment unit 14, and a housing 19 (refer to FIG. 2).

The temperature adjustment unit 14 heats and/or cools the sensitive unit 12.

The housing 19 houses the sensitive unit 12 and the temperature adjustment unit 14 and is removably attached to the electronic device 20.

A breath detection method performed by the breath detection system 1 includes a temperature control step and an instruction information output step.

The temperature control step includes controlling the temperature of the sensitive unit 12 to cause the temperature of the sensitive unit 12 to change in a predetermined temperature change pattern with time during a measuring period.

The instruction information output step includes outputting instruction information to instruct the subject how to blow his or her breath toward the sensitive unit 12 during an exhalation period. The exhalation period is set in accordance with the temperature change pattern.

As used herein, the expression “how to blow his or her breath toward the sensitive unit 12” refers to at least one selected from the group consisting of the timing to blow the breath toward the sensitive unit 12, the strength of the breath to be blown toward the sensitive unit 12, and the distance between the sensitive unit 12 and the subject's mouth (face). As used herein, the “instruction information” refers to a piece of information instructing the subject on at least one of the timing to blow the breath toward the sensitive unit 12, the strength of the breath to be blown toward the sensitive unit 12, or the distance between the sensitive unit 12 and the subject's mouth (face). That is to say, the instruction information preferably includes at least one piece of information selected from the group consisting of information about a period for which the subject blows his or her breath toward the sensitive unit 12, information about the strength of the breath, and information about the distance between the subject and the sensitive unit 12. The user interface included in the electronic device 20 is a device having the ability to present the subject the instruction information that has been output in the instruction information output step. The user information may include, for example, at least one of a display device that displays the instruction information as either characters or an image or a loudspeaker that emits the instruction information as a voice.

The breath detection system 1 according to this embodiment causes, in the temperature control step, the temperature of the sensitive unit 12 to change in a predetermined temperature change pattern with time. Then, the breath detection system 1 outputs, in the instruction information output step, instruction information instructing the subject how to blow his or her breath toward the sensitive unit 12 during the exhalation period to be set in accordance with the temperature change pattern. This allows the subject to blow his or her breath following the way to blow his or her breath as instructed by the instruction information. This allows the properties of the breath to be measured based on the output of the sensitive unit 12 in a state where the breath is blown toward the sensitive unit 12 in an appropriate way suitable to the temperature change pattern of the sensitive unit 12. Consequently, the breath detection method according to this embodiment may reduce the chances of measuring the subject's breath while allowing the subject to blow his or her breath inappropriately toward the sensitive unit 12.

### (2) Details

### (2.1) Configuration

As shown in FIG. 1, the breath detection system 1 according to this embodiment includes the sensor module 10 and the electronic device 20. The electronic device 20 may be, for example, a smartphone used by the subject.

### (2.1.1) Sensor Module

The sensor module 10 includes the sensitive unit 12, the temperature adjustment unit 14, and the housing 19 as described above. The sensor module 10 further includes a temperature sensor 13, a pressure sensor 15, a distance sensor 16, a humidity sensor 17, a control unit 11, and a communications unit 18.

The housing 19 houses the sensitive unit 12, the temperature adjustment unit 14, the temperature sensor 13, the pressure sensor 15, the distance sensor 16, the humidity sensor 17, the control unit 11, and the communications unit 18. That is to say, inside the housing 19, provided is a housing space (hereinafter also referred to as a “chamber”) 190 for housing at least the sensitive unit 12, the temperature adjustment unit 14, the temperature sensor 13, the pressure sensor 15, and the humidity sensor 17. The surface of the housing 19 is provided with a plurality of vent holes 191 that allows the housing space 190 to communicate with the external space outside of the housing 19. When the subject blows his or her breath, the breath passes through the vent holes 191 to enter the housing space 190.

Note that the housing 19 is provided with a connector 192 which may be connected to the electronic device's 20 connector compliant with the universal serial bus (USB) standard, for example. Connecting the connector 192 provided for the housing 19 to the connector of the electronic device 20 allows the housing 19 to be attached to the electronic device 20. In the state where the housing 19 is connected to the electronic device 20, electric power is supplied from the electronic device 20 to the sensor module 10. That is to say, the sensor module 10 is activated with the electric power supplied from the electronic device 20.

In this embodiment, the surface, provided with the display device 23, of the electronic device 20 will be hereinafter referred to as a “front surface.” In the state where the housing 19 is attached to the electronic device 20, the vent holes 191 are provided through the front surface of the housing 19.

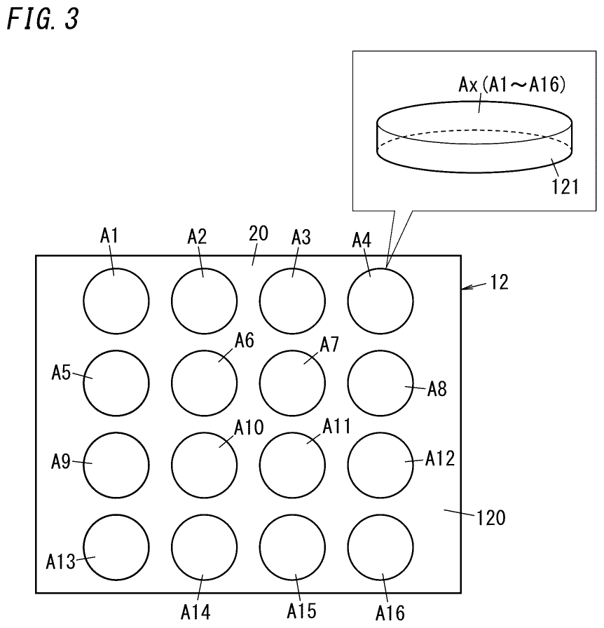

The sensitive unit 12 includes a plurality of sensitive elements Ax having mutually different sensitivities as shown in FIG. 3. In this embodiment, the sensitive unit 12 includes sixteen sensitive elements Ax, which will be hereinafter sometimes referred to as “sensitive elements A1-A16.” The sixteen sensitive elements A1-A16 are arranged in four rows and four columns on a board 120 having a flat plate shape. On the other principal surface of the board 120, provided is a heater for heating the sensitive elements A1-A16 arranged on the board 120. That is to say, in this embodiment, the temperature adjustment unit 14 is implemented as the heater disposed on the board 120.

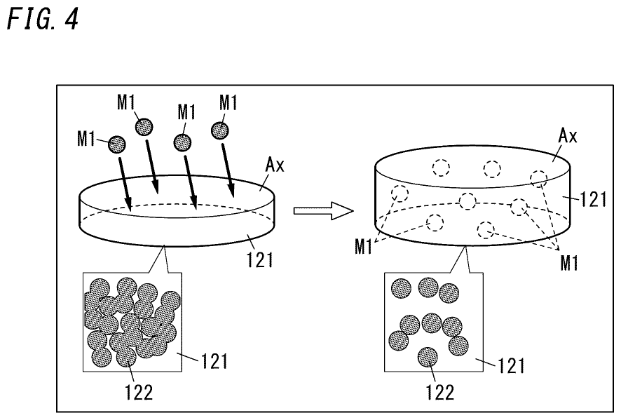

Each of the plurality of sensitive elements Ax included in the sensitive unit 12 includes: an organic composition 121 formed out of an organic material, having sensitivity to the molecules to detect which are included in the breath, into a disk shape; and conductive particles 122 dispersed in the organic composition 121. Each of the plurality of sensitive elements Ax is formed in a film shape as shown in FIGS. 3 and 4. When breath including the molecules to detect is blown toward the sensitive element Ax, the organic composition 121 absorbs the molecules to detect and expands. In FIG. 4, the portion on the left-hand side illustrates a state of the sensitive element Ax that has not absorbed the odor molecules M1 yet and the portion on the right-hand side illustrates a state of the sensitive element Ax that has absorbed the odor molecules M1. When the sensitive element Ax absorbs the odor molecules M1, the organic composition 21 expands. Thus, after the sensitive element Ax has absorbed the odor molecules M1, the interval between the conductive particles 122 widens, and electrical resistance as an electrical characteristic value of the sensitive element Ax increases, compared to the interval and the electrical resistance before the sensitive element Ax absorbs the odor molecules M1.

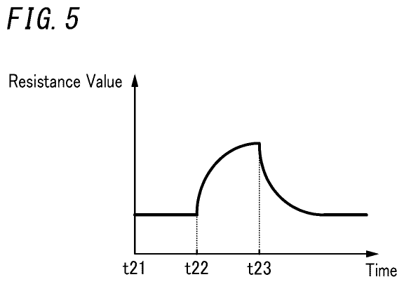

Note that the sensitive element Ax has temperature dependence that causes an electrical characteristic value (electrical resistance) thereof to vary according to the temperature. In this case, there are two types of sensitive elements Ax, namely, a sensitive element Ax, of which the electrical resistance increases as the temperature rises and which has a positive resistance coefficient and a sensitive element Ax, of which the electrical resistance decreases as the temperature rises and which has a negative resistance coefficient. FIG. 5 is a graph showing the resistance value of the sensitive element Ax having a positive resistance coefficient. In the period from a time t21 through a time t22, the temperature adjustment unit 14 stops supplying an electric current to the heater and the resistance value of the sensitive element Ax is constant. In the period from the time t22 through a time t23, as the temperature adjustment unit 14 supplies an electric current to the heater, the temperature of the sensitive element Ax rises and the resistance value increases accordingly. Thereafter, when the temperature adjustment unit 14 stops supplying the electric current to the heater at the time t23, the temperature of the sensitive element Ax falls due to natural air cooling and the resistance value decreases accordingly.

In this embodiment, the sensitive unit 12 includes negative characteristic sensitive elements having a negative resistance coefficient in a temperature range equal to or higher than −20° C. and equal to or lower than 50° C. For example, the sensitive elements A1-A11 correspond to negative characteristic sensitive elements. The sensitive unit 12 also includes positive characteristic sensitive elements having a positive resistance coefficient in a temperature range equal to or higher than −20° C. and equal to or lower than 50° C. For example, the sensitive elements A12-A16 correspond to positive characteristic sensitive elements.

The temperature sensor 13 may include, for example, a temperature measuring resistor, of which the resistance value changes according to the temperature. The temperature sensor 13 may be disposed, for example, in the housing space 190 to detect the temperature inside the housing space 190 as the temperature of the sensitive unit 12. The temperature sensor 13 outputs the detected temperature value of the sensitive unit 12 to the control unit 11.

The temperature adjustment unit 14 may include, for example, a heater provided on the board 120 on which the sensitive unit 12 is provided and a driver circuit for supplying an electric current to the heater. The temperature adjustment unit 14 controls the current value of the current flowing through the heater in accordance with a control signal supplied from the control unit 11, thereby either heating or cooling the sensitive unit 12. In this embodiment, the temperature adjustment unit 14 lowers the temperature of the sensitive unit 12 by natural air cooling by stopping the supply of the electric current to the heater. Optionally, the temperature adjustment unit 14 may heat and cool the sensitive unit 12 using a Peltier element, for example.

The pressure sensor 15 may include, for example, a piezoelectric element for transforming pressure into an electrical signal. The pressure sensor 15 is arranged in the housing space 190 to output a detection signal, representing the pressure inside the housing space 190 (i.e., the atmospheric pressure), to the control unit 11.

The distance sensor 16 is an ultrasonic sensor for measuring the distance to an object by a contactless method based on, for example, the time it takes an ultrasonic wave radiated from the distance sensor 16 and then reflected from the object to be received at the distance sensor 16. The distance sensor 16 is provided right under the openings of the vent holes 191 of the housing 19. This allows the distance sensor 16 to detect, when the subject blows his or her breath through the vent holes 191, the distance from the sensitive unit 12 to the subject's mouth (or face). The distance sensor 16 outputs the detection result of the distance to the control unit 11.

The humidity sensor 17 contains a humidity sensitive material, of which the resistance value changes when absorbing or desorbing humidity, for example, and outputs a detection signal representing the resistance value of the humidity sensitive material. The humidity sensor 17 is disposed in the housing space 190. When breath is blown into the housing space 190, the humidity sensor 17 outputs a detection signal corresponding to the humidity of the breath to the control unit 11. Alternatively, the humidity sensor 17 may contain a humidity sensitive material, of which the capacitance value changes responsive to absorption or desorption of humidity, and may output a detection signal representing the capacitance value of the humidity sensitive material.

The communications unit 18 communicates with the electronic device 20. The communications unit 18 may communicate with the electronic device 20 in accordance with a communications protocol compliant with the USB standard, for example. Note that the communication protocol applicable to the communication between the communications unit 18 and the electronic device 20 does not have to be compliant with the USB standard but may also be changed as appropriate.

The control unit 11 is a control circuit for controlling the overall operation of the sensor module 10. The control unit 11 may be implemented as, for example, a computer system including one or more processors (microprocessors) and one or more memories. That is to say, the functions of the control unit 11 are performed by making the one or more processors execute one or more programs (application software) stored in the one or more memories. The program may be stored in advance in, for example, an internal memory of the control unit 11. Alternatively, the program may also be downloaded via a telecommunications line such as the Internet or distributed after having been stored in a non-transitory storage medium such as a memory card.

The control unit 11 outputs an electrical characteristic value of the sensitive unit 12 to the electronic device 20 and controls the temperature adjustment unit 14 in accordance with the temperature control information provided by the electronic device 20. More specifically, during the measuring period, for example, the control unit 11 outputs a control signal to the temperature adjustment unit 14 in accordance with a temperature control signal that the communications unit 18 has received from the electronic device 20, thereby making the temperature adjustment unit 14 change the temperature of the sensitive unit 12 in a predetermined temperature change pattern. In addition, during the measuring period, the control unit 11 acquires a detection signal from the sensitive unit 12 and respective detection signals from the temperature sensor 13, the pressure sensor 15, the distance sensor 16, and the humidity sensor 17 at appropriate timings and outputs these control signals to the electronic device 20 via the communications unit 18.

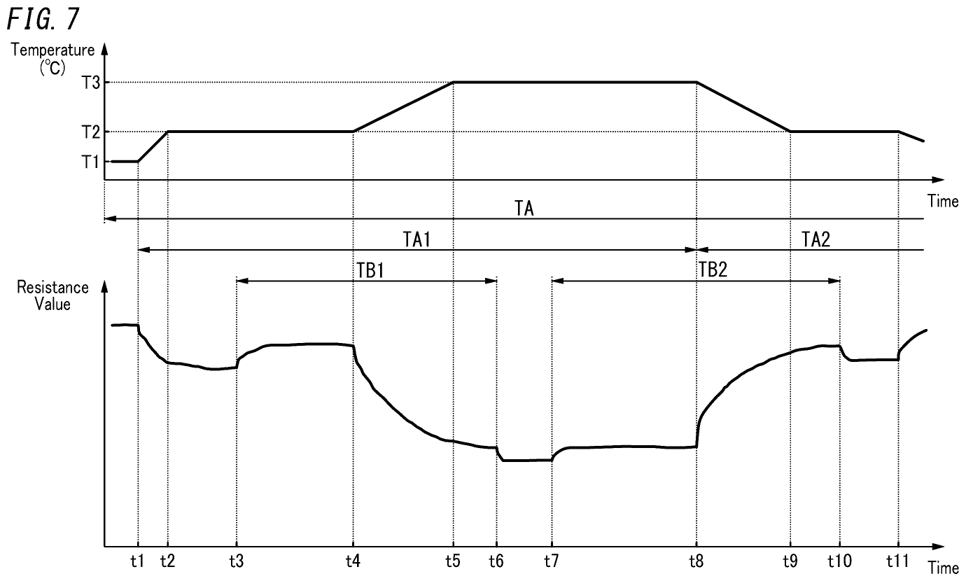

Referring to FIG. 7, the upper graph shows an exemplary temperature change pattern in which the temperature of the sensitive unit 12 is to be changed in the measuring period TA. The temperature change pattern of the sensitive unit 12 in the measuring period TA preferably includes at least a temperature rising period TA1 in which the temperature of the sensitive unit 12 rises and a temperature falling period TA2 in which the temperature of the sensitive unit 12 falls. More specifically, in the temperature rising period TA1, the temperature adjustment unit 14 controls the quantity of heat generated by the heater, thereby increasing the temperature of the sensitive unit 12 from a temperature T1 (which is a temperature when the sensitive unit 12 is not heated with the heater and may be 25° C., for example) to a temperature T2 (e.g., 40° C.) from a time t1 to a time t2. Once the temperature of the sensitive unit 12 reaches the temperature T2, the temperature adjustment unit 14 will maintain the temperature of the sensitive unit 12 at the temperature T2 until a time t4. Thereafter, the temperature adjustment unit 14 increases the temperature of the sensitive unit 12 from the temperature T2 to a temperature T3 (e.g., 70° C.) from the time t4 to a time t5. Once the temperature of the sensitive unit 12 reaches the temperature T3, the temperature adjustment unit 14 will maintain the temperature of the sensitive unit 12 at the temperature T3 until a time t8. In the temperature falling period TA2, the temperature adjustment unit 14 controls the quantity of heat generated by the heater, thereby decreasing the temperature of the sensitive unit 12 from the temperature T3 to the temperature T2 from the time t8 to a time t9. Once the temperature of the sensitive unit 12 reaches the temperature T2, the temperature adjustment unit 14 will maintain the temperature of the sensitive unit 12 at the temperature T2 until a time t11. Thereafter, after the time t11, the temperature adjustment unit 14 causes the heater to stop generating heat, thereby decreasing the temperature of the sensitive unit 12 from the temperature T2 to the temperature T1. Changing the temperature of the sensitive unit 12 in the predetermined temperature change pattern in the measuring period TA allows the breath detection system 1 to acquire the time series data of the output of the sensitive unit 12 in the temperature rising period TA1 and the time series data of the output of the sensitive unit 12 in the temperature falling period TA2. Consequently, this allows the breath detection system 1 to detect the properties of the breath more accurately based on the time series data of the outputs (electrical characteristic values) of the sensitive unit 12 in the temperature rising period TA1 and the temperature falling period TA2.

### (2.1.2) Electronic Device

The electronic device 20 forms, along with the sensor module 10, the breath detection system 1.

The electronic device 20 may be, for example, a smartphone used by the subject. In the electronic device 20, installed is a program (application software) designed to cause the electronic device 20 to perform the breath detection method according to this embodiment.

The electronic device 20 includes a control unit 21, a camera 22, a display device 23, a loudspeaker 24, a communications unit 25, and a storage unit 26.

The display device 23 may be, for example, a liquid crystal display and is installed on the front surface of the body 200 of the electronic device 20. The display device 23 may output instruction information for the subject in the form of characters or an image (which is either a still picture or a moving picture).

The loudspeaker 24 is built in the body 200. The loudspeaker 24 may output the instruction information for the subject as a voice.

The camera 22 is a front camera disposed on the front surface of the body 200. The camera 22 may shoot the mouth of the subject who blows his or her breath toward the vent holes 191 of the sensor module 10 attached to the body 200.

The communications unit 25 communicates with the sensor module 10. The communications unit 25 may communicate with the sensor module 10 in accordance with a communications protocol compliant with the USB standard, for example. Note that the communication protocol applied to the communication between the communications unit 25 and the sensor module 10 does not have to be compliant with the USB standard but may also be changed as appropriate.

The storage unit 26 includes one or more storage devices. Examples of the storage devices include a RAM, a ROM, and an EEPROM. The storage unit 26 stores, for example, a learned model for use to determine the properties of the breath. The learned model may be generated, for example, by making an artificial intelligence program (algorithm) machine-learn the relationship between training data and the properties of the breath. In this case, the training data may be, for example, the time series data of the output of the sensitive unit 12 when the subject blows his or her breath toward the sensitive unit 12 in the measuring period TA in which the temperature of the sensitive unit 12 is allowed to change in the predetermined temperature change pattern. The artificial intelligence program is a machine learning model. For example, a neural network, which is a type of a hierarchical model, may be used as an artificial intelligence program. The learned model may be generated by the breath detection system 1. Alternatively, the learned model may also be generated by a learning system other than the breath detection system 1.

The control unit 21 is a control circuit for controlling the overall operation of the electronic device 20. The control unit 21 may be implemented as, for example, a computer system including one or more processors (microprocessors) and one or more memories. That is to say, the functions of the control unit 21 are performed by making the one or more processors execute one or more programs (applications) stored in the one or more memories. The program may be stored in advance in, for example, an internal memory of the control unit 21. Alternatively, the program may also be downloaded via a telecommunications line such as the Internet or distributed after having been stored in a non-transitory storage medium such as a memory card.

The control unit 21 may have, for example, the functions of a temperature controller 31, an acquirer 32, an instruction information outputter 33, a display controller 34, a determiner 35, a breath determiner 36, and a decision result outputter 37. Note that the temperature controller 31, the acquirer 32, the instruction information outputter 33, the display controller 34, the determiner 35, the breath determiner 36, and the decision result outputter 37 only represent the respective functions to be performed by the control unit 21 and do not necessarily have a substantive physical configuration.

The temperature controller 31 outputs a temperature control signal to the sensor module 10 via the communications unit 25, thereby making the temperature adjustment unit 14 change the temperature of the sensitive unit 12. Specifically, the temperature controller 31 outputs a temperature control signal, which causes the temperature of the sensitive unit 12 to change in the predetermined temperature change pattern in the measuring period TA, to the sensor module 10, thereby making the temperature adjustment unit 14 change the temperature of the sensitive unit 12 in the predetermined temperature change pattern.

The acquirer 32 acquires, during the measuring period TA, breath related information about how the subject has blown his or her breath toward the sensitive unit 12. In this embodiment, the acquirer 32 generates image information representing the subject's face by subjecting the subject's face image, captured by the camera 22, to image processing, and acquires the subject's face image as a piece of the breath related information. Note that the subject's face image does not have to be an image generated by shooting his or her entire face but may be an image generated by shooting at least his or her mouth and surrounding regions. That is to say, the acquirer 32 performs an acquisition step including acquiring image information representing the subject's mouth as a piece of the breath related information by subjecting an image captured by the camera 22 that has shot the subject's face to image processing.

In addition, the acquirer 32 also acquires, as another piece of breath related information, a detected pressure value from the pressure sensor 15 of the sensor module 10 via the communications unit 25. That is to say, the acquirer 32 performs an acquisition step including acquiring, as another piece of breath related information, a detected pressure value from the pressure sensor 15 that detects an atmospheric pressure inside the housing chamber 190 in which the sensitive unit 12 is housed.

Besides, the acquirer 32 further acquires, as still another piece of breath related information, a detected distance value from the distance sensor 16 of the sensor module 10 via the communications unit 25. That is to say, the acquirer 32 performs an acquisition step including acquiring, as still another piece of breath related information, a detected distance value from the distance sensor 16 that detects the distance between the subject and the sensitive unit 12.

Besides, the acquirer 32 further acquires, as still another piece of breath related information, a detected humidity value from the humidity sensor 17 of the sensor module 10 via the communications unit 25. As can be seen, in this embodiment, the acquirer 32 acquires, as various pieces of breath related information, the subject's face image, the pressure value detected in the housing space 190, the detected distance value, and the humidity value detected in the housing space 190. Note that the acquirer 32 does not have to acquire all of these pieces of information but may acquire, as the breath related information, at least one selected from the group consisting of the subject's face image, the pressure value detected in the housing space 190, the detected distance value, and the humidity value detected in the breath.

The instruction information outputter 33 outputs instruction information instructing the subject how to blow his or her breath toward the sensitive unit 12 during the exhalation period to be set in accordance with the temperature change pattern. The instruction information outputter 33 performs an instruction information output step including outputting the instruction information instructing, in accordance with the image information, for example, the subject how to open his or her mouth. The instruction information outputter 33 also performs an instruction information output step including outputting the instruction information instructing, in accordance with the pressure value detected by the pressure sensor 15, the subject how strongly the subject is supposed to blow his or her breath. In this case, the instruction information outputter 33 preferably outputs the instruction information about the strength of the breath to blow to allow the pressure value detected by the pressure sensor 15 to fall within a predetermined pressure range (permissible range). In addition, the instruction information outputter 33 further performs an instruction information output step including outputting the instruction information instructing, in accordance with the detected distance value, the subject on the distance between the subject and the sensitive unit 12 to allow the distance between the subject and the sensitive unit 12 to fall within a predetermined distance range.

FIG. 7 is a graph showing how the temperature of the sensitive unit 12 and the resistance value of a sensitive element Ax may change with time during the measuring period TA in which the breath is measured. In this embodiment, the temperature change pattern (refer to FIG. 7) of the sensitive unit 12 during the measuring period TA includes the temperature rising period TA1 in which the temperature of the sensitive unit 12 is raised and the temperature falling period TA2 in which the temperature of the sensitive unit 12 is lowered. Thus, the exhalation period includes a first exhalation period TB1 which is set during the temperature rising period TA1 and a second exhalation period TB2 including the temperature falling period TA2. The first exhalation period TB1 is set to begin at the time t3 when the temperature of the sensitive unit 12 is stabilized at the temperature T2 and end at the time t6 when the temperature of the sensitive unit 12 has increased to the temperature T3 to stabilize the output of the sensitive unit 12. The second exhalation period TB2 is set to begin at the time t7 when the output of the sensitive unit 12 is stabilized after the first exhalation period TB1 is over and to end at the time t10 when the temperature of the sensitive unit 12 has decreased to the temperature T2 to stabilize the output of the sensitive unit 12. Optionally, the instruction information outputter 33 may change the specifics of the instruction information in accordance with the breath related information acquired by the acquirer 32.

The display controller 34 has any desired information displayed on the screen of the display device 23 by outputting image data to the display device 23. The display controller 34 generates, in accordance with the instruction information provided by the instruction information outputter 33, the image data to be output to the display device 23 to have the instruction information provided by the instruction information outputter 33 displayed on the screen of the display device 23.

The determiner 35 determines whether the subject has blown his or her breath toward the sensitive unit 12. Specifically, the determiner 35 determines, based on the respective detection values acquired by the acquirer 32 from the pressure sensor 15 and the humidity sensor 17, for example, whether the subject has blown his or her breath toward the sensitive unit 12.

The breath determiner 36 infers the properties of the breath by entering, during the measuring period TA, the time series data of the output of the sensitive unit 12 when the temperature of the sensitive unit 12 is allowed to change in the predetermined temperature change pattern into a learned model. That is to say, the breath determiner 36 is in charge of an inference phase in which the properties of the breath are inferred based on the learned model.

The decision result outputter 37 outputs the result of the decision made by the breath determiner 36. The decision result outputter 37 outputs the result of the decision made by the breath determiner 36 by, for example, displaying the result of the decision made by the breath determiner 36 on the display device 23.

### (2.2) How Breath Detection System Works

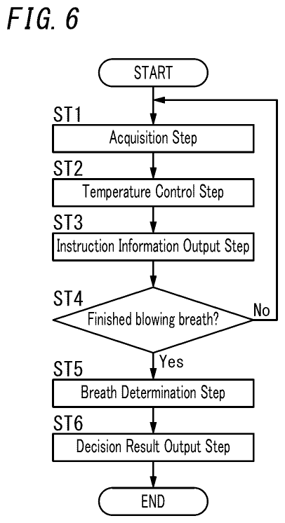

Next, it will be described with reference to the flowchart shown in FIG. 6 and other drawings how the breath detection system 1 according to this embodiment performs the breath detection operation. Note that the flow shown in FIG. 6 is only an exemplary procedure of the odor gas detection method according to this embodiment and should not be construed as limiting. Optionally, the processing steps shown in FIG. 6 may be performed in a different order from the illustrated one, some of the processing steps shown in FIG. 6 may be omitted as appropriate, and/or an additional processing step may be performed as needed.

The subject operates the electronic device 20 to run a breath detection program, installed in the electronic device 20, with the sensor module 10 connected to the electronic device 20. In response, the electronic device 20 starts performing a series of processing steps for detecting his or her breath.

The acquirer 32 of the electronic device 20 performs the acquisition step including acquiring the breath related information (in Step ST1). More specifically, the acquirer 32 subjects the image generated by the camera 22 to image processing, thereby extracting the subject's face image from the image generated by the camera 22 and acquiring the subject's face image information as a piece of the breath related information. In this case, the acquirer 32 preferably acquires, as the image information representing the subject's face, image information representing how the subject opens his or her mouth (including the shape and dimensions of the mouth). In addition, the acquirer 32 also acquires, from the sensor module 10, respective detection values of the pressure sensor 15 and the distance sensor 16 via the communications unit 25. Optionally, the acquirer 32 may further acquire, as still another piece of breath related information, the detection value of the humidity sensor 17.

Next, the temperature controller 31 of the electronic device 20 performs the temperature control step including outputting the temperature control signal that causes the temperature of the sensitive unit 12 to change in a predetermined temperature change pattern to the sensor module 10 via the communications unit 25 (in Step ST2). On receiving the temperature control signal via the communications unit 18, the control unit 11 of the sensor module 10 outputs a control signal to the temperature adjustment unit 14 to change the temperature of the sensitive unit 12 in the predetermined temperature change pattern.

Then, the instruction information outputter 33 output the instruction information instructing the subject how to blow his or her breath toward the sensitive unit 12 during the exhalation period (including the first exhalation period TB1 and the second exhalation period TB2 described above) to be set in accordance with the temperature change pattern (in Step ST3). In this embodiment, the display controller 34 creates, in accordance with the instruction information provided by the instruction information outputter 33, on-screen image data indicating specific instruction for the subject and output the on-screen image data to the display device 23. As a result, an on-screen instruction image indicating specific instructions for the subject is displayed on the display device 23.

FIG. 2 illustrates an exemplary on-screen instruction image displayed on the display device 23. On the upper part of the on-screen instruction image, displayed are the character “measuring” indicating that the breath is being measured. On the middle part of the on-screen instruction image, displayed is the subject's face image P1 shot by the camera 22. In this example, a mark Mk1 indicating ideal mouth shape and dimensions that would turn the breath blowing state into a desired one in accordance with the instruction information indicating the shape and dimensions of the mouth as provided by the instruction information outputter 33 is superimposed on the face image P1.

On the on-screen instruction image, a progress bar B1 indicating the measuring period TA and a character string W1 representing, in characters, the exhalation period during which the subject is supposed to blow his or her breath are displayed between the character “measuring” and the face image P1. The progress bar B1 indicates, on a single bar, where the current point in time is between the beginning and end of the measuring period TA. The cursor CS1 indicates the current point in time during the measuring period TA. The cursor CS1 moves to the right as time passes by. The character string W1 may be, for example, a character string such as “phew, phew” representing a breath blowing state. While the subject is blowing no breath, the characters are displayed in open letters.

On the on-screen instruction image, further displayed under the face image P1 are an indicator B2 instructing the pressure with which the subject is supposed to blow his or her breath and another indicator B3 instructing the subject how distant the subject is supposed to hold his or her face. These indicators B2 and B3 are vertically displayed one on top of the other.

The indicator B2 indicates, on a bar, the pressure with which the subject is blowing his or her breath. On the indicator B2, displayed are a mark Mk2 indicating the permissible pressure range and a cursor CS2 indicating the measured value of the pressure sensor 15. In the example illustrated in FIG. 2, the measured value of the pressure sensor 15 is short of the permissible range, and therefore, the instruction information outputter 33 displays an alert message “Blow your breath more strongly” over the indicator B2. That is to say, the instruction information outputter 33 outputs the instruction information about how strongly the subject needs to blow his or her breath to allow the pressure value detected by the pressure sensor 15 to fall within a predetermined pressure range (permissible range). Alternatively, the instruction information outputter 33 may also output the instruction information about the strength of the breath to blow to allow the pressure difference between the pressure value detected before the subject blows his or her breath and the pressure value detected while the subject is blowing his or her breath to fall within a predetermined pressure range.

The indicator B3 indicates, on the bar, the distance between the face of the subject who is blowing his or her breath and the sensitive unit 12. On the indicator B3, displayed are a mark Mk3 indicating a permissible distance range and a cursor CS3 indicating the measured value of the distance sensor 16. In the example illustrated in FIG. 2, the measured value of the distance sensor 16 is short of the permissible range (indicating that the subject's face is too close to the sensitive unit 12), and therefore, the instruction information outputter 33 displays an alert message “Hold your face more distant” over the indicator B3.

As can be seen, the display device 23 of the electronic device 20 displays an on-screen instruction image providing the instruction information instructing, either in characters or as an image, the subject how to blow his or her breath toward the sensitive unit 12. This allows the subject to blow his or her breath appropriately toward the sensitive unit 12 while watching the on-screen instruction image.

When the subject finishes blowing his or her breath (if the answer is YES in Step ST4), the breath determiner 36 determines the properties of the breath by entering the time series data of the output of the sensitive unit 12 during the measuring period TA into the learned model (in Step ST5). When the determination process is over, the decision result outputter 37 outputs the decision result of the properties of the breath (in Step ST6). For example, the decision result of the properties of the breath may be displayed on the display device 23 by, for example, making the display controller 34 generate on-screen data indicating the decision result provided by the decision result outputter 37 and output the on-screen data to the display device 23.

For example, if the breath determiner 36 determines, as the properties of the breath, whether the breath has any odor or whether the odor of the breath is good or bad, then the decision result outputter 37 may display, on the display device 23, a decision result about the odor of the breath. On the other hand, if the breath determiner 36 determines, as the properties of the breath, whether or not the concentration of alcohol in the breath is greater than a reference value, then the decision result outputter 37 may display, on the display device 23, a decision result about whether or not the concentration of alcohol in the breath is greater than the reference value.

### (3) Variations

Note that the embodiment described above is only an exemplary one of various embodiments of the present disclosure and should not be construed as limiting. Rather, the exemplary embodiment may be readily modified in various manners depending on a design choice or any other factor without departing from the scope of the present disclosure. Also, the functions of the breath detection system 1 may also be implemented as a breath detection method to be performed by the breath detection system 1, a computer program, or a non-transitory storage medium on which the program is stored. A breath detection method according to an aspect includes a temperature control step (Step ST2) and an instruction information output step (Step ST3). The temperature control step includes controlling the temperature of a sensitive unit 12 to cause the temperature of the sensitive unit 12 to change in a predetermined temperature change pattern with time during a measuring period TA. The instruction information output step includes outputting instruction information to instruct the subject how to blow his or her breath toward the sensitive unit 12 during an exhalation period (including a first exhalation period TB1 and a second exhalation period TB2). The exhalation period is set in accordance with the temperature change pattern. The breath detection method preferably further includes an acquisition step (Step ST1) including acquiring, during the measuring period TA, breath related information about how the subject has blown his or her breath toward the sensitive unit 12. The instruction information output step includes outputting the instruction information created based on the breath related information. Optionally, the breath detection method may further include: a breath determination step (Step ST5) including determining properties of the breath based on an electrical characteristic value of the sensitive unit 12 during the measuring period TA; and a decision result output step (Step ST6) including outputting a decision result of the breath determination step. A (computer) program according to another aspect is designed to cause a computer system to perform the breath detection method described above.

Next, variations of the exemplary embodiment will be enumerated one after another. Note that the variations to be described below may be adopted in combination as appropriate. In the following description of variations, any constituent element, having the same function as a counterpart of the exemplary embodiment described above, will be designated by the same reference numeral as that counterpart's, and description thereof will be omitted herein.

The breath detection system 1 according to the present disclosure or the agent that performs the breath detection method according to the present disclosure includes a computer system. The computer system may include a processor and a memory as principal hardware components thereof. The computer system performs the functions of the breath detection system 1 according to the present disclosure or serves as the agent that performs the breath detection method according to the present disclosure by making the processor execute a program stored in the memory of the computer system. The program may be stored in advance in the memory of the computer system. Alternatively, the program may also be downloaded through a telecommunications line or be distributed after having been recorded in some non-transitory storage medium such as a memory card, an optical disc, or a hard disk drive, any of which is readable for the computer system. The processor of the computer system may be made up of a single or a plurality of electronic circuits including a semiconductor integrated circuit (IC) or a large-scale integrated circuit (LSI). As used herein, the “integrated circuit” such as an IC or an LSI is called by a different name depending on the degree of integration thereof. Examples of the integrated circuits such as an IC or an LSI include integrated circuits called a “system LSI,” a “very-large-scale integrated circuit (VLSI),” and an “ultra-large-scale integrated circuit (ULSI).” Optionally, a field-programmable gate array (FPGA) to be programmed after an LSI has been fabricated or a reconfigurable logic device allowing the connections or circuit sections inside of an LSI to be reconfigured may also be adopted as the processor. Those electronic circuits may be either integrated together on a single chip or distributed on multiple chips, whichever is appropriate. Those multiple chips may be aggregated together in a single device or distributed in multiple devices without limitation. As used herein, the “computer system” includes a microcontroller including one or more processors and one or more memories. Thus, the microcontroller may also be implemented as a single or a plurality of electronic circuits including a semiconductor integrated circuit or a large-scale integrated circuit.

In the embodiment described above, the plurality of functions of the breath detection system 1 are aggregated together in a single housing. However, this is not an essential configuration for the breath detection system 1. Alternatively, those constituent elements of the breath detection system 1 may be distributed in multiple different housings. Still alternatively, at least some functions of the breath detection system 1 (e.g., some functions of the breath detection system 1) may be implemented as a cloud computing system as well. For example, the breath determiner 36 may determine the properties of the breath by using a learned model put on the cloud computing system. Specifically, the breath determiner 36 of the breath detection system 1 may enter the time series data provided by the sensitive unit 12 during the measuring period TA into the learned model on the cloud computing system and acquire the decision result from the learned model on the cloud computing system to determine the properties of the breath.

Also, in the exemplary embodiment described above, the functions of the sensor module 10 may be installed in the electronic device 20.

Furthermore, in the exemplary embodiment described above, the control unit 21 may perform the humidity detection step including detecting, based on the detection value acquired from the humidity sensor 17 via the communications unit 25, the humidity of the breath that the subject blows toward the sensitive unit 12. Then, the determiner 35 may perform, based on the result of detection of the humidity in the humidity detection step, the determination step including determining whether the subject has blown his or her breath toward the sensitive unit 12. The determiner 35 may compare the result of detection of the humidity in the humidity detection step with a predetermined humidity range, for example. When finding the detection result of the humidity lower or higher than the predetermined humidity range, the determiner 35 decides that the subject not have blown his or her breath toward the sensitive unit 12. If the determiner 35 decides that the subject not have blown his or her breath in the exhalation period that is set during the measuring period TA, then the instruction information outputter 33 outputs instruction information instructing that the measurement be redone. This allows the properties of the breath to be detected with the subject's breath blown appropriately toward the sensitive unit 12.

Furthermore, in the exemplary embodiment described above, the instruction information outputter 33 outputs, as pieces of instruction information, information about a period during which the subject is supposed to blow his or her breath toward the sensitive unit 12, information about the strength of the breath, and information about the distance between the subject and the sensitive unit 12. However, the instruction information outputter 33 does not have to output all of these pieces of information. Alternatively, the instruction information outputter 33 may output, as the instruction information, one or more pieces of information selected from the group consisting of the information about the period during which the subject is supposed to blow his or her breath toward the sensitive unit 12, the information about the strength of the breath, and the information about the distance between the subject and the sensitive unit 12. Still alternatively, the instruction information outputter 33 may also output any other piece of information.

Furthermore, in the exemplary embodiment described above, the breath determiner 36 may perform the processing of identifying, based on a component pattern of the breath, the person who has blown his or her breath. The control unit 21 of the electronic device 20 may use the result of the person identification by the breath determiner 36 to authenticate the user when the application software is started. This allows only an authorized user to run the application software.

Also, in the breath detection system 1 according to the embodiment described above, the sensitive unit 12 includes sixteen sensitive elements Ax. However, the number of the sensitive elements Ax provided may be changed as appropriate. The sensitive unit 12 includes both negative characteristic sensitive elements and positive characteristic sensitive elements. However, this is only an example and should not be construed as limiting. Alternatively, the sensitive unit 12 may include only negative characteristic sensitive elements or only positive characteristic sensitive elements. Furthermore, in the breath detection system 1 according to the embodiment described above, the sixteen sensitive elements Ax are arranged in four columns and four rows. However, the plurality of sensitive elements Ax do not have to be arranged in the arrangement pattern described for the exemplary embodiment. Alternatively, the plurality of sensitive elements may also be arranged in line. Still alternatively, the plurality of sensitive elements may also be arranged at intervals to form a single circular pattern or a plurality of concentric circular pattern.

In the exemplary embodiment described above, the electronic device 20 to which the sensor module 10 is connected does not have to be a smartphone. Rather, the electronic device 20 only needs to include a user interface for presenting the instruction information to the subject. Thus, the electronic device 20 may also be a tablet computer or a dedicated measuring device, whichever is appropriate.

### Recapitulation

The exemplary embodiment and its variations described above are specific implementations of the following aspects of the present disclosure.

A breath detection method according to a first aspect includes a temperature control step and an instruction information output step. The temperature control step includes controlling a temperature of a sensitive unit (12) to cause the temperature of the sensitive unit (12) to change in a predetermined temperature change pattern with time during a measuring period. The instruction information output step includes outputting instruction information to instruct a subject how to blow a breath toward the sensitive unit (12) during an exhalation period. The exhalation period is set in accordance with the temperature change pattern.

This aspect may reduce the chances of measuring a subject's breath while allowing the subject to blow the breath inappropriately toward the sensitive unit (12).

In a breath detection method according to a second aspect, which may be implemented in conjunction with the first aspect, the instruction information includes at least one piece of information selected from the group consisting of: information about a period in which the subject blows the breath toward the sensitive unit (12); information about a strength of the breath; and information about a distance between the subject and the sensitive unit (12).

This aspect may reduce, by outputting instruction information about at least one of a period in which the subject blows the breath, the strength of the breath, or a distance between the subject and the sensitive unit (12), the chances of measuring the subject's breath while allowing the subject to blow the breath inappropriately toward the sensitive unit (12).

A breath detection method according to a third aspect, which may be implemented in conjunction with the first or second aspect, further includes an acquisition step. The acquisition step includes acquiring breath related information about how the subject has blown the breath toward the sensitive unit (12) during the measuring period. The instruction information output step includes outputting the instruction information created based on the breath related information.

This aspect allows instruction information to be output based on the breath related information about how the subject has blown the breath during the measuring period.

In a breath detection method according to a fourth aspect, which may be implemented in conjunction with the third aspect, the acquisition step includes acquiring image information representing the subject's mouth as a piece of the breath related information by subjecting an image captured by a camera (22) to image processing. The camera (22) is arranged to shoot the subject's face. The instruction information output step includes outputting the instruction information instructing, in accordance with the image information, the subject how to open a mouth.

This aspect allows for outputting the instruction information instructing, in accordance with the image information representing the subject's mouth, the subject how to open the mouth.

In a breath detection method according to a fifth aspect, which may be implemented in conjunction with the third or fourth aspect, the acquisition step includes acquiring, as another piece of the breath related information, a detected pressure value from a pressure sensor (15). The pressure sensor (15) detects an atmospheric pressure inside a chamber (190) in which the sensitive unit (12) is housed. The instruction information output step includes outputting the instruction information instructing, in accordance with the detected pressure value, the subject how strongly the subject is supposed to blow the breath.

This aspect allows for outputting the instruction information instructing, in accordance with the pressure value detected by the pressure sensor (15), the subject how strongly the subject is supposed to blow the breath.

In a breath detection method according to a sixth aspect, which may be implemented in conjunction with the fifth aspect, the instruction information output step includes outputting the instruction information about how strongly the subject is supposed to blow the breath to allow the detected pressure value to fall within a predetermined pressure range.

This aspect allows for instructing the subject how strongly the subject is supposed to blow the breath to cause the detected pressure value to fall within a predetermined pressure range.

In a breath detection method according to a seventh aspect, which may be implemented in conjunction with the fifth aspect, the instruction information output step includes outputting the instruction information about how strongly the subject is supposed to blow the breath to allow a pressure difference between the pressure value detected before the subject starts to blow the breath and the pressure value detected while the subject is blowing the breath to fall within a predetermined pressure range.

This aspect allows for instructing the subject how strongly the subject is supposed to blow the breath to allow a pressure difference between the pressure value detected before the subject starts to blow the breath and the pressure value detected while the subject is blowing the breath to fall within a predetermined pressure range.

In a breath detection method according to an eighth aspect, which may be implemented in conjunction with any one of the third to seventh aspects, the acquisition step includes acquiring, as the breath related information, a detected distance value from a distance sensor (16). The distance sensor (16) detects a distance between the subject and the sensitive unit (12). The instruction information output step includes outputting the instruction information instructing, in accordance with the detected distance value, the subject on the distance between the subject him- or herself and the sensitive unit (12) to allow the distance between the subject and the sensitive unit (12) to fall within a predetermined distance range.

This aspect allows for outputting the instruction information instructing, in accordance with the distance value detected by the distance sensor (16), the subject on the distance between the subject him- or herself and the sensitive unit (12).

A breath detection method according to a ninth aspect, which may be implemented in conjunction with any one of the first to eighth aspects, further includes a humidity detection step and a determination step. The humidity detection step includes detecting a humidity of the breath that the subject blows toward the sensitive unit (12). The determination step includes determining, based on a result of detection of the humidity in the humidity detection step, whether the subject has blown the breath toward the sensitive unit (12). The instruction information output step includes outputting the instruction information instructing, in accordance with a decision made in the determination step, the subject how the subject is supposed to blow the breath.

This aspect allows for determining, based on a result of detection of the humidity, whether or not the subject has blown the breath toward the sensitive unit (12), and outputting the instruction information in accordance with the decision made about whether the subject has blown the breath.

In a breath detection method according to a tenth aspect, which may be implemented in conjunction with any one of the first to ninth aspects, the temperature change pattern includes at least a temperature rising period in which the temperature of the sensitive unit (12) rises and a temperature falling period in which the temperature of the sensitive unit (12) falls.

This aspect allows for acquiring time series data provided by the sensitive unit (12) in a situation where the subject has blown the breath toward the sensitive unit (12) with the temperature of the sensitive unit (12) caused to change in a temperature change pattern including at least a temperature rising period and a temperature falling period.

A breath detection method according to an eleventh aspect, which may be implemented in conjunction with any one of the first to tenth aspects, further includes a breath determination step and a decision result output step. The breath determination step includes determining properties of the breath based on an electrical characteristic value of the sensitive unit (12) during the measuring period. The decision result output step includes outputting a decision result of the breath determination step.

This aspect allows the properties of the breath to be determined based on an electrical characteristic value of the sensitive unit (12) in a situation where the temperature of the sensitive unit (12) is caused to change in the predetermined temperature pattern.

A program according to a twelfth aspect is designed to cause a computer system to perform the breath detection method according to any one of the first to eleventh aspects.

This aspect may reduce the chances of measuring a subject's breath while allowing the subject to blow the breath inappropriately toward the sensitive unit (12).

A sensor module (10) according to a thirteenth aspect is connectible to an electronic device. The electronic device includes a user interface and is designed to instruct a subject how to blow a breath toward a sensitive unit (12). The sensor module (10) includes: the sensitive unit (12); a temperature adjustment unit (14) to heat and/or cool the sensitive unit (12); and a housing (19) which houses the sensitive unit (12) and the temperature adjustment unit (14) and is designed to be removably attached to the electronic device (20).

This aspect may reduce the chances of measuring a subject's breath while allowing the subject to blow the breath inappropriately toward the sensitive unit (12).

A sensor module (10) according to a fourteenth aspect, which may be implemented in conjunction with the thirteenth aspect, further includes a control unit (11) that outputs an electrical characteristic value of the sensitive unit (12) to the electronic device (20) and controls the temperature adjustment unit in accordance with temperature control information provided by the electronic device (20).

This aspect may reduce the chances of measuring a subject's breath while allowing the subject to blow the breath inappropriately toward the sensitive unit (12).

Note that these are not the only aspects of the present disclosure but various configurations (including variations) of the breath detection system (1) according to the exemplary embodiment described above may also be implemented as, for example, a breath detection method, a (computer) program, or a non-transitory storage medium on which the program is stored.

Note that the features according to the second to eleventh aspects are not essential features for the breath detection method but may be omitted as appropriate. Note that the constituent element according to the fourteenth aspect is not an essential constituent element for the sensor module (10) but may be omitted as appropriate.

### REFERENCE SIGNS LIST

10 Sensor Module
11 Control Unit
12 Sensitive Unit
14 Temperature Adjustment Unit
15 Pressure Sensor
16 Distance Sensor
19 Housing
20 Electronic Device
22 Camera
190 Chamber

1. A breath detection method comprising:
a temperature control step including controlling a temperature of a sensitive unit to cause the temperature of the sensitive unit to change in a predetermined temperature change pattern with time during a measuring period; and
an instruction information output step including outputting instruction information to instruct a subject how to blow a breath toward the sensitive unit during an exhalation period, the exhalation period being set in accordance with the temperature change pattern.

2. The breath detection method of claim 1, wherein
the instruction information includes at least one piece of information selected from the group consisting of: information about a period in which the subject blows the breath toward the sensitive unit; information about a strength of the breath; and information about a distance between the subject and the sensitive unit.

3. The breath detection method of claim 1, further comprising an acquisition step including acquiring breath related information about how the subject has blown the breath toward the sensitive unit during the measuring period, wherein
the instruction information output step includes outputting the instruction information created based on the breath related information.

4. The breath detection method of claim 3, wherein
the acquisition step includes acquiring image information representing the subject's mouth as a piece of the breath related information by subjecting an image captured by a camera to image processing, the camera being arranged to shoot the subject's face, and
the instruction information output step includes outputting the instruction information instructing, in accordance with the image information, the subject how to open a mouth.

5. The breath detection method of claim 3, wherein
the acquisition step includes acquiring, as another piece of the breath related information, a detected pressure value from a pressure sensor, the pressure sensor being configured to detect an atmospheric pressure inside a chamber in which the sensitive unit is housed, and
the instruction information output step includes outputting the instruction information instructing, in accordance with the detected pressure value, the subject how strongly the subject is supposed to blow the breath.

6. The breath detection method of claim 5, wherein the instruction information output step includes outputting the instruction information about how strongly the subject is supposed to blow the breath to allow the detected pressure value to fall within a predetermined pressure range.

7. The breath detection method of claim 5, wherein
the instruction information output step includes outputting the instruction information about how strongly the subject is supposed to blow the breath to allow a pressure difference between the pressure value detected before the subject starts to blow the breath and the pressure value detected while the subject is blowing the breath to fall within a predetermined pressure range.

8. The breath detection method of claim 3, wherein
the acquisition step includes acquiring a detected distance value from a distance sensor as the breath related information, the distance sensor being configured to detect a distance between the subject and the sensitive unit, and
the instruction information output step includes outputting the instruction information instructing, in accordance with the detected distance value, the subject on the distance between the subject him- or herself and the sensitive unit to allow the distance between the subject and the sensitive unit to fall within a predetermined distance range.

9. The breath detection method of claim 1, further comprising:
a humidity detection step including detecting a humidity of the breath that the subject blows toward the sensitive unit; and
a determination step including determining, based on a result of detection of the humidity in the humidity detection step, whether the subject has blown a breath toward the sensitive unit, wherein
the instruction information output step includes outputting the instruction information instructing, in accordance with a decision made in the determination step, the subject how the subject is supposed to blow the breath.

10. The breath detection method of claim 1, wherein
the temperature change pattern includes at least a temperature rising period in which the temperature of the sensitive unit rises and a temperature falling period in which the temperature of the sensitive unit falls.

11. The breath detection method of claim 1, further comprising:
a breath determination step including determining properties of the breath based on an electrical characteristic value of the sensitive unit during the measuring period; and
a decision result output step including outputting a decision result of the breath determination step.

12. A computer-readable, non-transitory, and tangible recording medium recording a program to be stored in a computer, the program causing a computer system to perform the breath detection method of claim 1.

13. A sensor module connectible to an electronic device, the electronic device including a user interface and configured to instruct a subject how to blow a breath toward a sensitive unit, the sensor module comprising:
the sensitive unit;
a temperature adjustment unit configured to heat and/or cool the sensitive unit; and
a housing which houses the sensitive unit and the temperature adjustment unit and is designed to be removably attached to the electronic device.

14. The sensor module of claim 13, further comprising a control unit configured to output an electrical characteristic value of the sensitive unit to the electronic device and control the temperature adjustment unit in accordance with temperature control information provided by the electronic device.

15. The breath detection method of claim 2, further comprising an acquisition step including acquiring breath related information about how the subject has blown the breath toward the sensitive unit during the measuring period, wherein
the instruction information output step includes outputting the instruction information created based on the breath related information.

16. The breath detection method of claim 4, wherein
the acquisition step includes acquiring, as another piece of the breath related information, a detected pressure value from a pressure sensor, the pressure sensor being configured to detect an atmospheric pressure inside a chamber in which the sensitive unit is housed, and
the instruction information output step includes outputting the instruction information instructing, in accordance with the detected pressure value, the subject how strongly the subject is supposed to blow the breath.

17. The breath detection method of claim 4, wherein
the acquisition step includes acquiring a detected distance value from a distance sensor as the breath related information, the distance sensor being configured to detect a distance between the subject and the sensitive unit, and
the instruction information output step includes outputting the instruction information instructing, in accordance with the detected distance value, the subject on the distance between the subject him- or herself and the sensitive unit to allow the distance between the subject and the sensitive unit to fall within a predetermined distance range.

18. The breath detection method of claim 5, wherein
the acquisition step includes acquiring a detected distance value from a distance sensor as the breath related information, the distance sensor being configured to detect a distance between the subject and the sensitive unit, and
the instruction information output step includes outputting the instruction information instructing, in accordance with the detected distance value, the subject on the distance between the subject him- or herself and the sensitive unit to allow the distance between the subject and the sensitive unit to fall within a predetermined distance range.

19. The breath detection method of claim 6, wherein
the acquisition step includes acquiring a detected distance value from a distance sensor as the breath related information, the distance sensor being configured to detect a distance between the subject and the sensitive unit, and
the instruction information output step includes outputting the instruction information instructing, in accordance with the detected distance value, the subject on the distance between the subject him- or herself and the sensitive unit to allow the distance between the subject and the sensitive unit to fall within a predetermined distance range.

20. The breath detection method of claim 7, wherein
the acquisition step includes acquiring a detected distance value from a distance sensor as the breath related information, the distance sensor being configured to detect a distance between the subject and the sensitive unit, and
the instruction information output step includes outputting the instruction information instructing, in accordance with the detected distance value, the subject on the distance between the subject him-or herself and the sensitive unit to allow the distance between the subject and the sensitive unit to fall within a predetermined distance range.
